(19) United States
(12) Patent Application Publication
(10) Pub. No.: US 2007/0156223 A1
(43) Pub. Date: Jul. 5, 2007

(54) Stent delivery system with improved delivery force distribution

(76) Inventor: Dennis Vaughan, North Miami, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/322,968

(22) Filed: Dec. 30, 2005

(51) Int. Cl. A61F 2/06

(52) U.S. Cl. 623/1.11

(57) ABSTRACT

A stent delivery system that helps equalize delivery forces throughout a vascular prosthesis during delivery thereof to an intended treatment site. The delivery system comprises a catheter having a lumen therethrough, an inner core over which the one or more stents or stent grafts is mounted for delivery from the catheter, and an outer sheath overlying the one or more stent or stent graft and the inner core. The inner core of the further comprises a series of outwardly flared members that engage segments or fill voids in or between the stent, stents or stent grafts, respectively. The outwardly flared members of the inner core thus help maintain the segments of the stent or the one or more stents or stent grafts in spaced relation with respect to one another as delivery thereof occurs and as removal of the outer sheath occurs to position the one or more stents or stent grafts at the intended treatment site. The outwardly flared members thus help to distribute delivery forces more evenly throughout the one or more stents or stent grafts being delivered from the catheter. The outwardly flared members further aid accurate placement of the one or more stents or stent grafts by maintaining longitudinal spacing thereof during delivery.

### BACKGROUND OF THE INVENTION

1. Field of the Invention

The invention generally relates to a stent delivery system. More specifically, the invention relates to a stent delivery system that improves emplacement of one or more stents or stent grafts by better distributing delivery forces during delivery thereof to an intended treatment site.

2. Related Art

Balloon expandable stents and self-expanding stents are generally delivered in a cylindrical form, compressed to a smaller diameter, and placed within a vessel using a catheter-based delivery system. A femoral artery in the groin is typically accessed to deliver the stent graft to its intended position in the abdominal region, for example, the abdominal aortic artery. A guidewire is threaded from the femoral artery access and through the blood vessel to a point beyond the diseased part of the artery.

The delivery system, carrying the stent or stent graft within it, is manipulated through the blood vessel and over the guidewire to a position beyond the diseased area of the blood vessel. Fluoroscopy (x-ray imaging) is typically used to help guide the delivery system and the stent or stent graft to the intended treatment site. Once the stent or stent graft is located as desired, an outer sheath of the delivery system is gradually withdrawn to enable the stent or stent graft to expand and anchors itself to the inside of the vessel. Some stent or stent grafts require inflation of a balloon in order to expand within the vessel. Other stent grafts are comprised of self-expanding materials that expand upon withdrawal thereof from within the delivery system sheath. Hooks or barbs at ends, or other positions, of the stent may be used to help anchor the stent to the vessel walls.

Typically, delivery of the stent or stent graft requires a push rod, or other member, that engages a proximal end of the stent or stent graft during deployment thereof from the delivery catheter. Although engaging the proximal end of the stent or stent graft as the sheath is withdrawn helps to maintain the proximal end of the stent or stent graft in place, such engagement may also cause more distal segments of the stent or stent graft, or adjacent stents or stent grafts, to buckle with one another.

To overcome the above cited buckling of adjacent segments, stents or stent grafts, some delivery systems incorporate a release wire that anchors to a distal most segment of a stent or stent graft. The release wire thus maintains the segments, stent or stent graft in the appropriate alignment relative to one another even as the outer sheath is withdrawn or the push rod is engaged against a proximal end of the stent or stent graft. After deployment, the release wire is independently withdrawn through the catheter. Although the release wire tends to provide good placement of a stent or stent graft, such a release wire configuration increases the steps necessary for delivering a stent or stent graft to an intended treatment site and may limit the reduction of delivery catheter French size due to inclusion of such a release wire and components associated therewith. Moreover, a release wire delivery system tends to be costly and more complicated to manufacture,

In view of the above, a need exists for systems and methods that simplifies delivery of a stent or stent graft to an intended treatment site, and that better distributes delivery forces throughout a stent or stent graft during delivery thereof, while minimizing the costs and manufacturing complexity of the delivery system.

### SUMMARY OF THE INVENTION

The invention comprises a stent or stent graft delivery system for use with one or more expandable stents or stent grafts. The one or more expandable stents or stent grafts may comprise a series of adjacent segments having voids therebetween such segments of a single stent or stent graft, or may comprise one or more adjacent stents or stent grafts having voids between such adjacent stents or stent grafts. In any case, the delivery system comprises a catheter having a lumen therethrough, an inner core over which the one or more stents or stent grafts is mounted for delivery from the catheter, and an outer sheath overlying the one or more stent or stent graft and the inner core. Whereas conventional stent delivery systems comprise an independent push rod that engages a proximal end of the stent to be delivered, the inner core of the delivery system described herein further comprises a series of members that flare outwardly from the inner core and towards the outer sheath to engage segments or fill voids in or between the stent, stents or stent grafts, respectively. The outwardly flared members of the inner core thus help maintain the segments of the stent or the one or more stents or stent grafts in spaced relation with respect to one another as delivery thereof through and catheter and removal of the outer sheath occurs to position the one or more stents or stent grafts at the intended treatment site. The outwardly flared members thus help to distribute delivery forces more evenly throughout the one or more stents or stent grafts being delivered from the catheter.

In some embodiments, the outwardly flared members are integrally molded with the inner core and increasingly taper proximally to distally such that the smaller diametered proximal portion of each flared member eventually blends into the inner core, whereas the larger diametered distal portion of each flared member extend to engage segments of the single stent or to fill or engage respective voids and engage portions of the one or more stents or stent grafts during delivery thereof. Ideally, the larger diametered distal portion of the outwardly flared members engage a proximal portion of each adjacent stent or stent graft to maximize the distribution of delivery forces thereto the one or more stents or stent grafts. In other embodiments, the flared segmented members are axially slit to comprise collapsible fingers, the ends of which fill or engage the respective segments, voids and portions of the one or more stents or stent grafts during delivery thereof. In either case, a biocompatible, lubricious material preferably comprises the inner core and the outwardly flared members. Each successive flared member of the series of flared members is similarly configured. This configuration provides an inner member with sufficient columnar strength to maintain the one or more stents or stent grafts in spaced relation to one another during delivery thereof, and to effect delivery of the one or more stents or stent grafts through the catheter to the intended treatment site without the need for more complex release wires or other instruments or steps. This configurations also enables easy removal of the inner core after deployment of the one or more stents or stent grafts has occurred at the intended treatment site as a result of the lesser diametered proximal portion of each flared member leading the withdrawal of the inner core from the patient after the one or more stents or stent grafts has resumed its expanded state and disengaged the larger diametered flared member portions from the one or more stent or stent graft.

The above and other features of the invention, including various novel details of construction and combinations of parts, will now be more particularly described with reference to the accompanying drawings and claims. It will be understood that the various exemplary embodiments of the invention described herein are shown by way of illustration only and not as a limitation thereof. The principles and features of this invention may be employed in various alternative embodiments without departing from the scope of the invention.

### BRIEF DESCRIPTION OF THE DRAWINGS

These and other features, aspects, and advantages of the apparatus and methods of the present invention will become better understood with regard to the following description, appended claims, and accompanying drawings where:

FIG. 1 illustrates an exemplary conventional catheter-based stent delivery system.

FIG. 2 illustrates an embodiment of a catheter-based stent delivery system according to the invention.

FIG. 3 illustrates a single stent mounted onto an inner core of the delivery system of FIG. 2 according to the invention.

FIG. 4 illustrates multiple stents mounted onto an inner core of the delivery system of FIG. 2 according to the invention.

FIG. 5 illustrates a stent graft mounted onto an inner core of the delivery system of FIG. 2 according to the invention.

FIGS. 6A & 6B illustrates another embodiment of the inner core according to the invention.

FIG. 7 illustrates an example of outwardly flared members integrally molded with the inner core according to various embodiments of the invention described herein.

### DETAILED DESCRIPTION OF THE INVENTION

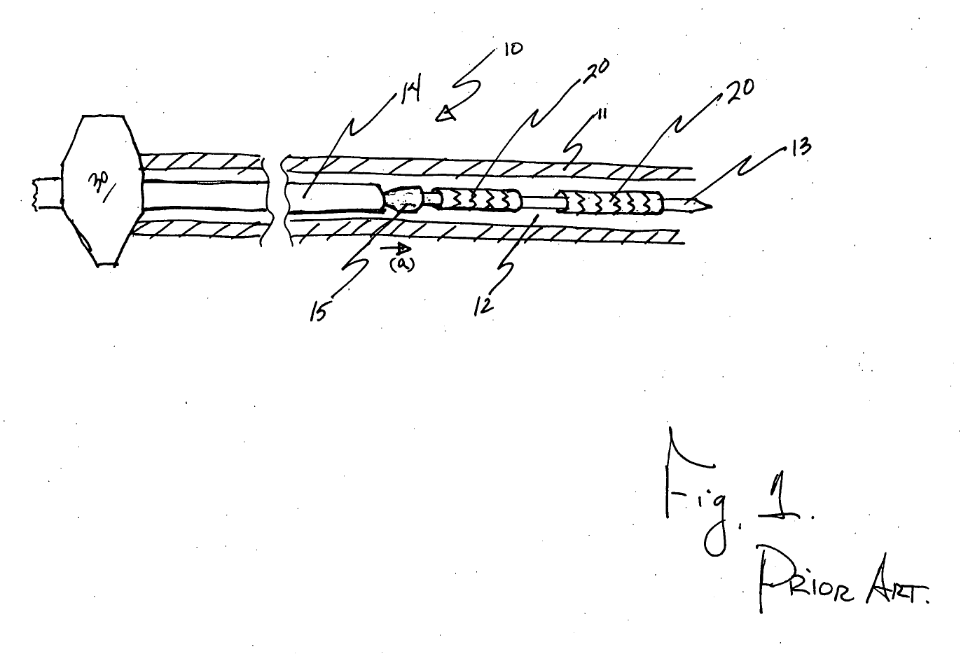

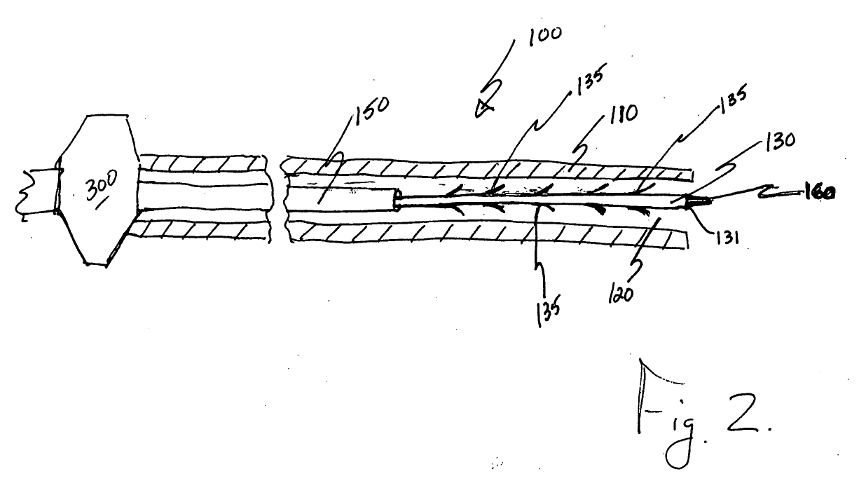

FIG. 1 illustrates a conventional catheter-based stent delivery system 10 having one or more stents 20 delivered therefrom to an intended treatment site. As shown in FIG. 1, the conventional stent delivery system 10 comprises a catheter 11 having a lumen 12 extending therethrough, an inner member 13, an outer sheath 14, and a pushrod 15. A hub 30 may be provided to help the operator handle insertion and withdrawal thereof. The pushrod 15 is moved in the direction of arrow (a) in order to maintain the one or more stents 20 in place as the outer sheath 14 is withdrawn to deploy the stents 20 as desired, or the pushrod is moved in the direction of arrow (a) in order to help push the one or more stents 20 into the desired deployed position. In either case, the use of the pushrod 14 against the proximal end of the one or more stents 20 tends to buckle the stents 20 or otherwise alter the spacing within or between the one or more stents 20. The altered spacing often results in an undesirable positioning of the one or more stents 20 relative to the intended treatment site. Moreover, where buckling within the one or more stents 20 occurs, such buckling risks injury to the vasculature of the patient and typically requires redeployment of the one or more stents 20. Buckling often also requires the use of new stents to replace the damaged or buckled stents 20 so as to reduce the risk of injury to the patient and to increase the efficacy of the one or more stents at the intended treatment site.

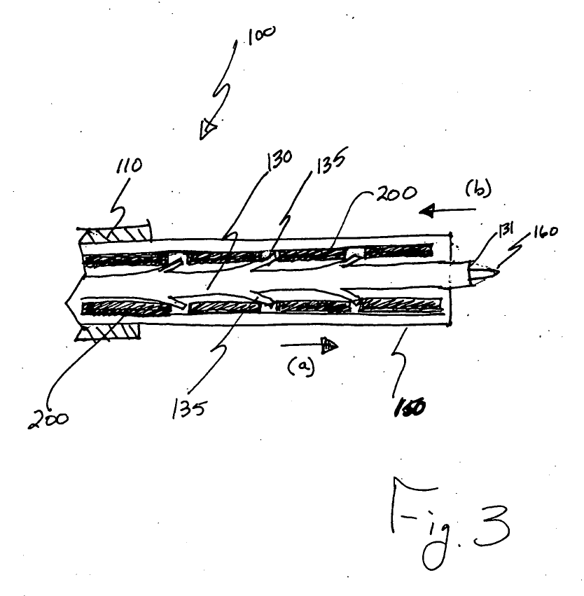

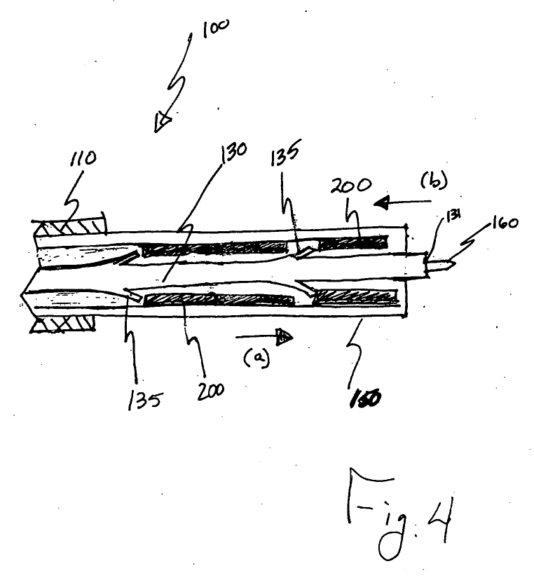

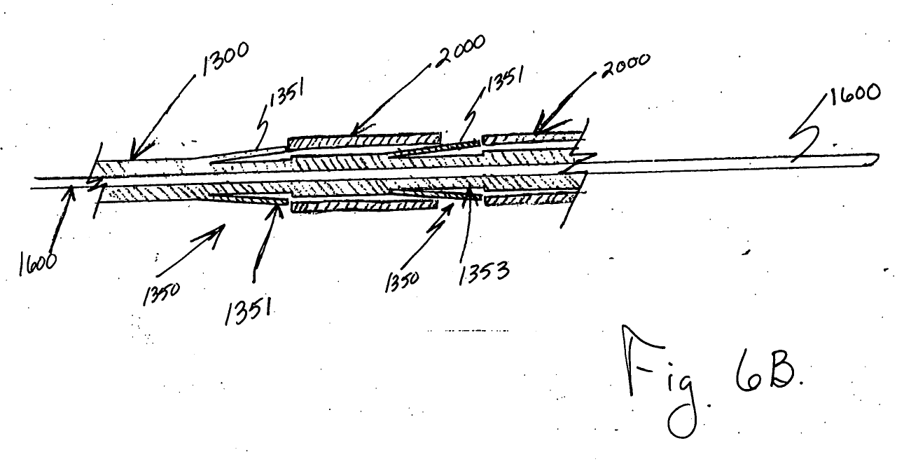

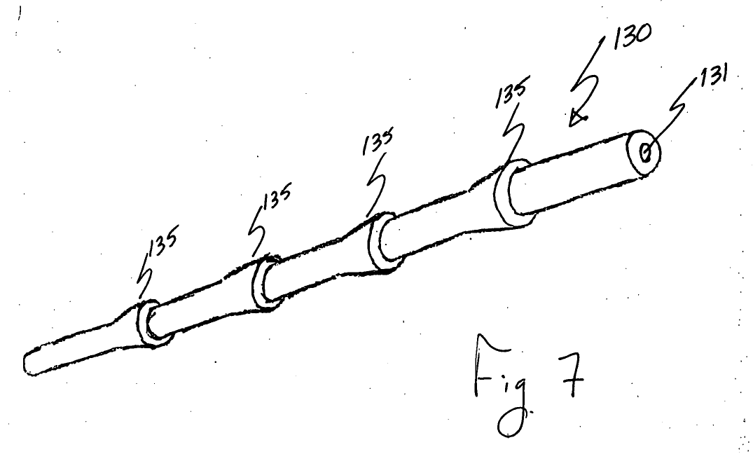

FIG. 2 illustrates an embodiment of a catheter-based stent delivery system 100 according to the invention described herein. As shown in FIG. 2, the stent delivery system 100 comprises a catheter 110 with a lumen 120 extending therethrough, an inner core 130, and an outer sheath 150. The inner core 130 further comprises a central lumen 131 extending through the inner core 130 and through which lumen 131 a guidewire 160 is provided to help locate the catheter 110 as desired. The inner core 130 further comprises a series of outwardly flared members 135 extending outwardly from the inner core 130. The outwardly flared members 135 may be integral with the inner core 130 as shown in FIGS. 3 and 7, for example, or the outwardly flared members 1350 may be integral with the inner core 1300 as a series of collapsible fingers 1351, as in FIGS. 6A & 6B, for example.

Referring again to FIG. 2, a hub 300 may be provided to help insertion and withdrawal of the system 100 by the operator. One or more stents or stent grafts 200 are mounted onto the inner core 130 (FIG. 3) for delivery therefrom the stent delivery system 100. Each outwardly flared member 135 of the series of outwardly flared members 135 comprises a larger diametered distal portion and a smaller diametered proximal portion, wherein distal is understood herein to refer to locations furthest from the medical operator and proximal is understood to refer to locations closest to the medical operator. The smaller diametered proximal portion of each flared member 135 generally blends into the inner core 130, and the larger diametered distal portion of each flared member 135 engages a segment or a portion of the one or more stents or stent grafts mounted on the inner core 130 for eventual delivery therefrom.

FIG. 3 illustrates a single stent 200 mounted, in its crimped state, onto an inner core 130 of the stent delivery system 100 of FIG. 2, for example. The larger diametered distal portion of the outwardly flared members 135 of the inner core 130 project through voids in the stent 200 to engage axially adjacent segments of the stent 200. The outwardly flared member 135 may include axial slits so as to comprise collapsible fingers 1351 from the outwardly flared member as in FIGS. 6A & 6B, for example, wherein the outwardly flared members are identified as 1350. Referring still to FIGS. 6A & 6B, the outwardly flared member 1350 having collapsible fingers 1351 permit some of the collapsible fingers 1351 to project through voids in the stent 2000 to engage segments of the stent 2000 during, while others of the collapsible fingers 1351 are confined from engaging the stent 2000 in the same manner by solid portions of the stent. The engagement of the axially adjacent segments of the stent 200, or 2000, in this manner by the flared members 135, or collapsible fingers 1351 of flared members 1350, help to maintain the spatial orientation of the stent and segments thereof as delivery of the stent is effected.

In particular, referring again to FIG. 3, after mounting of the stent 200 onto the inner core 130 as described above, delivery of the stent 200 is effected by movement of the inner core 130 in the direction of arrow (a), whereafter the outer sheath 150 is withdrawn in the direction of arrow (b) when the stent 200 has reached the intended treatment site. Upon withdrawal of the outer sheath 140 by movement in the direction of arrow (b), the expandable stent 200 resumes its expanded state within the blood vessel, thereby disengaging the stent 200 from the larger diametered portions of the respective flared members 135. The inner core 130 is thereafter withdrawn by movement thereof in the direction of arrow (b), while the stent 200 remains deployed within the blood vessel at the intended treatment site.

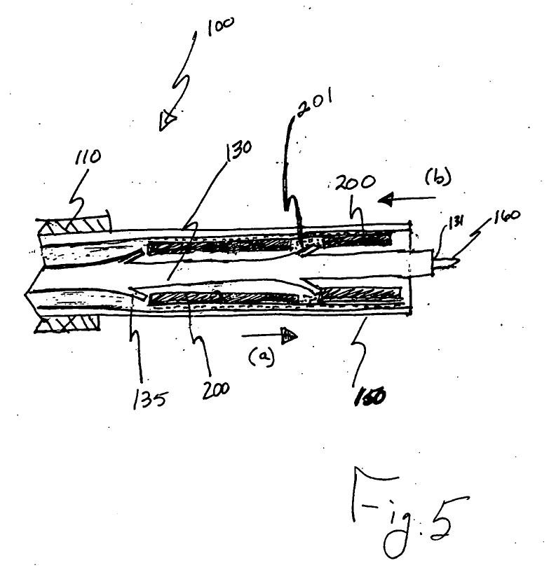

Although the mounting and delivery of a single stent 200 is described above, the artisan will readily appreciate that a series of one or more stents or stent grafts may be instead, or additionally, be mounted onto the inner core 130 for delivery from the stent delivery system 100 otherwise described above. The multiple stents 200 may be the same size and length, or may be different sizes or lengths, in order to accommodate different physiological needs in the discretion of the medical practitioner. Spacing between the outwardly flared members 135 on the inner core 130 may be varied to accommodate the different sizes or lengths of multiple stents, or the inner core 130 with one spacing arrangement between the outwardly flared members 135 may be interchangeably used with variously lengthed or sized stents. In any event, where multiple stents 200 are mounted for delivery from the stent delivery system 100, the outwardly flared members 135 of the inner core ideally engage proximal ends of the stents 200, although engagement of other portions of the stents 200 through voids therein, may also be used. Engagement of the multiple stents 200 in this manner helps to distribute delivery forces more equally among the stents 200, while maintaining the spatial orientation of the stents 200 as delivery thereof is effected. FIG. 4 illustrates an example of multiple stents 200 mounted, in their crimped state, on an inner core 130 as described above. Delivery thereof the multiple stents 200 is otherwise effected as described above with respect to a single stent 200, as the artisan will readily appreciate.

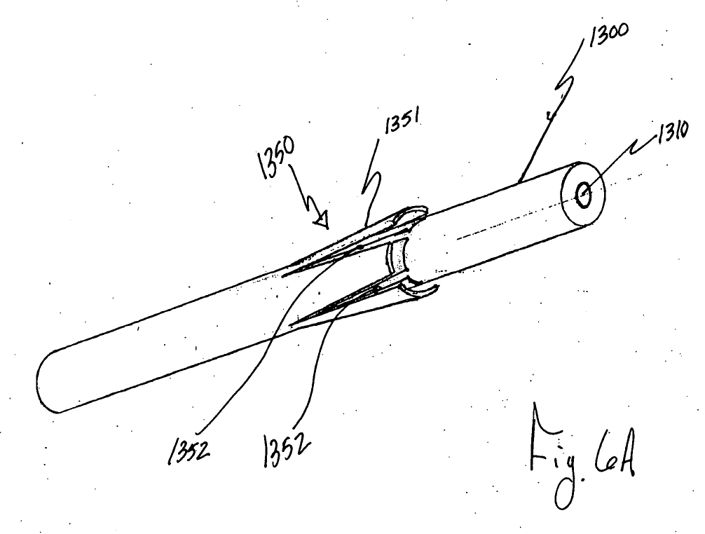

FIG. 5 illustrates the case where the stent is a stent graft 200 having graft material 201 associated therewith. Where one or more stent grafts 200 is deployed via the stent delivery system 100 described herein, the stent grafts 200 are mounted to the inner core 130 as in earlier described embodiments. When crimped onto the inner core 130, the outwardly flared members 135 displace the graft material 201 otherwise existing in the voids of the stent graft whereat the outwardly flared members 135 engage portions of the stent graft 200 for delivery from the catheter-based delivery system 100. Ideally, as before, the outwardly flared members 135 engage the proximal ends of the one or more stent grafts 200 in order to help maintain the spatial orientation of the stent graft 200 and to help distribute delivery forces more evenly throughout the stent graft 200 as the stent graft is delivered to the intended treatment site. In practice, the delivery of the one or more stent grafts 200 using the catheter-based delivery system having the inner core 130 and series of outwardly flared members 135 described herein is as described above, as the artisan will readily appreciate. Thus, a single stent graft, or multiple. stent grafts, may be delivered similar to the delivery of a single stent, or multiple stents, as otherwise described above.

Preferably, the inner core 130 is a solid tubular piece having the outwardly flared members 135 made integrally therewith. The inner core 130 and outwardly flared members 135 are comprised of a biocompatible, lubricious material, such as Pebax, for example. Of course, the artisan will appreciate that other known or later developed biocompatible, lubricious materials may also comprise the inner core 130 and outwardly flared members 135 instead of, or in addition to, the Pebax, for example. Such other materials may comprise biocompatible plastics, polymers, or blends thereof, for example. Because imbedment of the inner core 130 or outwardly flared members 135 into the one or more stents or stent grafts, or segments thereof, is not required according to the delivery system and techniques described herein, a compressible material is not required to comprise the inner core or outwardly flared members.

Although described above in the context of a solid inner core 130, FIGS. 6A & 6B illustrates aspects of an alternative embodiment of an inner core 1300 usable with the delivery system otherwise described herein. In particular, FIG. 6A illustrates the inner core 1300 having outwardly flared members 1350 along portions thereof. Although only one outwardly flared member is shown in FIG. 6A, the artisan will readily appreciate that an inner core 1300 may also be comprised of a series of similarly configured outwardly flared members 1350. FIG. 6B, for example, shows in cross-section, an inner core 1300 comprised of a series of outwardly flared members 1350 having collapsible fingers 1351 engaging portions of one or more stents or stent grafts 2000. The outwardly flared members 1350 further comprise collapsible fingers 1351 formed by axial slits 1352 provided in the outwardly flared members. The axial slits 1352 generally extend from a distal tip of the outwardly flared member to the outer surface of the inner core 1300. In this manner, the collapsible fingers 1351 are able to accommodate engagement of a variety of stent or stent graft designs, whereby some of the collapsible fingers 1351 will project through voids in the stent or stent graft 2000 to engage segments thereof for delivery, whereas others of the collapsible fingers 1351 will be confined from engaging segments of the stent or stent graft 2000 in the same manner by solid portions of the stent or stent graft. The collapsible fingers 1351 thus flex inwardly away from the one or more stents or stent grafts 2000 mounted thereon the inner core 1300 to reduce delivery forces experienced during delivery, which generally occurs as otherwise described above. The flexible nature of the collapsible fingers 1351 helps the inner core 1300 more readily conform to irregularities exhibited in the interior diameter of the one or more stents or stent grafts during delivery, or during crimping thereof onto the inner core 1300. As shown in FIGS. 6A & 6B, the collapsible fingers 1351 also flex inwardly into recesses 1353 formed in the inner core 1300 during delivery of the one or more stents or stent grafts 2000, which also helps minimize the profile thereof during delivery.

The various exemplary embodiments of the invention as described hereinabove do not limit different embodiments of the systems and methods of the invention. The material described herein is not limited to the materials, designs or shapes referenced herein for illustrative purposes only, and may comprise various other materials, designs or shapes suitable for the systems and methods described herein, as should be appreciated by the artisan.

While there has been shown and described what is considered to be preferred embodiments of the invention, it will, of course, be understood that various modifications and changes in form or detail could readily be made without departing from the spirit or scope of the invention. It is therefore intended that the invention be not limited to the exact forms described and illustrated herein, but should be construed to cover all modifications that may fall within the scope of the appended claims.

What is claimed is:

1. A stent delivery system for use with expandable stents, the stent delivery system comprising:
a catheter having a lumen extending therethrough;
an inner core having a single stent or one or more adjacent stents or stent grafts crimped thereon for delivery therefrom;
a series of outwardly flared members extending from the inner member and engaging segments of the single stent or portions of the one or more adjacent stents or stent grafts during delivery thereof; and
an outer sheath overlying the single stent or one or more stents or stent grafts, and the inner core.

2. The stent delivery system of claim 1, wherein the outwardly flared members are integrally molded with the inner core.

3. The stent delivery system of claim 1, wherein each outwardly flared member increasingly tapers proximally to distally such that each further comprises a larger diametered distal portion and a smaller diametered proximal portion, the smaller diametered proximal portion generally blending into the inner core.

4. The stent delivery system of claim 1, wherein engagement of the segments, adjacent stents, or adjacent stent grafts by the series of outwardly flared members distributes delivery force more uniformly and maintains spacing of the stent, stents or stent grafts during delivery thereof to an intended treatment site.

5. The stent delivery system of claim 1, wherein the outwardly flared members further comprises axial slits and collapsible fingers formed thereby.

6. The stent delivery system of claim 5, wherein the axial slits extend from a distal end of the outwardly flared member towards, but not beyond, the inner core.

7. The stent delivery system of claim 5, wherein the inner core further comprises recessed areas into which the collapsible fingers may flex to lower the delivery profile of the one or more stents or stent grafts.

8. A method for delivering one or more stents or stent grafts via a catheter-based delivery system, the method comprising:
mounting one or more expandable stents or stent graft in a crimped state to an inner core of the catheter-based delivery system;
engaging outwardly flared members of the inner core to portions of the one or more stents or stent grafts to maintain the one or more stents in a desired spatial orientation during delivery thereof to an intended treatment site;
disposing an outer sheath over the one or more stents or stent grafts;
inserting the delivery system to the intended treatment site;
distributing delivery forces throughout the one or more stents or stent grafts during delivery thereof by the engagement of the outwardly flared members with the portions of the one or more stents or stent grafts; and
withdrawing the outer sheath and resuming an expanded state of the one or more stents thereby disengaging the outwardly flared members from the one or more stents or stent grafts; and
withdrawing the inner core.

9. The method of claim 8, further comprising axially slitting portions of the outwardly flared members from a distal end thereof towards the inner core of the catheter-based delivery system.

10. The method of claim 9, further comprising forming collapsible fingers from the outwardly flared members by the axially slitting thereof.

11. The method of claim 10, wherein engaging the outwardly flared members further comprises engaging some of the collapsible fingers with the portions of the one or more stent or stent grafts by penetration of the some of the collapsible fingers through voids in the one or more stents or stent grafts, while confining others of the collapsible fingers from such engagement due to solid portions of the one or more stent or stent grafts.

12. The method of claim 11, further comprising flexing the collapsible fingers into recesses in the inner core to minimize a delivery profile of the one or more stents or stent grafts.
